(19) United States
(12) Patent Application Publication
(10) Pub. No.: US 2015/0141875 A1
(43) Pub. Date: May 21, 2015

(54) COMPOSITIONS AND METHODS FOR NON-INVASIVE DETECTION AND TREATMENT OF HYPOXIC CELLS IN VIVO

(71) Applicant: THE BOARD OF TRUSTEES OF THE UNIVERSITY OF ARKANSAS, Little Rock, AR (US)

(72) Inventors: Nathan A. Koonce, Little Rock, AR (US); Robert Griffin, Little Rock, AR (US)

(21) Appl. No.: 14/408,249

(22) PCT Filed: Jun. 14, 2013
(86) PCT No.: PCT/US13/45867

§ 371 Date: Dec. 15, 2014

Related U.S. Application Data
(60) Provisional application No. 61/659,832, filed on Jun. 14, 2012.

(51) Int. Cl. A61K 49/22; A61K 41/00; A61N 7/02; A61K 51/12

(52) U.S. Cl. CPC A61K 49/223; A61K 49/221; A61K 51/1203; A61K 41/0028; A61N 7/02; A61N 2007/0039
USPC 601/3; 424/9.52; 424/178.1; 424/400; 424/1.11

(57) ABSTRACT

Compositions and methods used in the non-invasive detection and/or treatment of hypoxic tissues in vivo are described. Compositions including microbubbles functionalized with one or more hypoxia targeting agents and one or more therapeutic compounds, methods of preparing the functionalized microbubbles, and methods of using the functionalized microbubbles for diagnostic and/or therapeutic applications are described, including a method for selectively determining the amount of vascular hypoxia occurring in a tissue.

### CROSS-REFERENCE TO RELATED APPLICATIONS

This application claims priority to U.S. Provisional Application Ser. No. 61/659,832 filed on Jun. 14, 2012 and entitled “Compositions and Methods for Non-Invasive Detection and Treatment of Hypoxic Cells In vivo”, the disclosure of which is hereby incorporated by reference in its entirety.

### FIELD OF THE INVENTION

The present disclosure relates to compositions used in the non-invasive detection and/or treatment of hypoxic tissues in vivo. In particular, the present disclosure relates to compositions comprising gas-filled microbubbles functionalized with one or more hypoxia targeting agents, methods of preparing the functionalized microbubbles, and methods of using the functionalized microbubbles for diagnostic and/or therapeutic applications.

### BACKGROUND

An ongoing challenge in oncology is the identification and elimination of treatment-resistant cells. Hypoxic cells, well-studied and treatment-resistant cells, are strongly associated with certain tumors, vascular diseases, and brain tissue regions afflicted by stroke. Diagnosis and treatment of such conditions may be enhanced by determining the extent and degree of hypoxia in the affected tissues of individual patients.

As certain tumors enlarge, the tumor tissue may outgrow its oxygen and nutrient supply, and at any given time a tumor may produce viable hypoxic cells. Hypoxic cells in solid tumors may be highly resistant to killing by some forms of chemotherapy. Because hypoxic tissue lacks a fully functioning blood supply network, the chemotherapeutic drugs may never reach the hypoxic cells. The presence of hypoxic cells may also impede radiation treatment because their low oxygen concentration renders the ionizing radiation relatively ineffective in killing the cancerous cells. Therefore, hypoxic cells are more likely to survive chemotherapy and radiation therapy and eventually lead to the reappearance of the tumor.

The vascular endothelium is ideally situated to respond to changes in local oxygenation and mediate physiological and pathological vascular responses to hypoxia. The oxygenation state of the endothelial cells represents a useful biomarker for tumor growth and metastasis. Tumor vascularization may be an important factor influencing radiotherapy response, and hypoxic vasculature may attenuate the desired effects of therapy as well as stimulate unwanted signaling in disease. Vascular hypoxia is a phenomenon strongly associated with tumor vasculature. Endothelial cells may have a greater resistance to radiation under both aerobic and hypoxic conditions than tumor cells.

A need exists for a method of detecting the presence of hypoxic cells associated with a tumor or surrounding vasculature within a patient in a predictable and non-invasive manner. Such a method would provide diagnostic, therapeutic and treatment planning adjuvants for human cancer and other disease states, without the concomitant hazards associated with radioactivity.

### SUMMARY

In one aspect, a composition for the detection of at least one hypoxic cell is provided. The composition includes a plurality of functionalized microbubbles. Each functionalized microbubble may include a microbubble with an exposed microbubble surface, a linking compound attached to the microbubble surface, and a selective binding compound attached to the linking compound opposite to the microbubble surface. In particular, the binding compound may selectively bind to a hypoxia-targeting agent attached to the hypoxic cell. The composition further functions as a targeted contrast agent for the detection of at least one hypoxic cell. The microbubble may have a microbubble diameter ranging from about 0.1 μm to about 10 μm. The linking compound may be chosen from: streptaviden, avidin, neutravidin, and any combination thereof. The selective binding compound may be chosen from: an antibody with an antigenic determinant that is the hypoxia-targeting agent; a polypeptide; a non-peptide molecule; and any combination thereof. The linking compound may be streptaviden and the selective binding compound is the antibody; the antibody may be conjugated with biotin and the antibody may be attached to the microbubble surface via a biotin-streptaviden linkage. The hypoxia-targeting agent may be chosen from an endogenous hypoxic cell marker and an exogenous hypoxia-targeting agent; the hypoxia-targeting agent may preferentially bind to the hypoxic cell. The hypoxia-targeting agent may be a compound, derivative, or metabolite of a compound chosen from: nitromidazole, misonidazole, pimonidazole, and any combination thereof. The at least one hypoxic cell may be detected using an imaging method chosen from: ultrasound, MRI, x-ray scattering, and PET. The composition may also function as a targeted treatment for the at least one hypoxic cell. Each functionalized microbubble may further include at least one therapeutic compound. Each therapeutic compound may be attached to the microbubble surface, attached to the linking compound, attached to the selective binding compound, or contained within the microbubble. Each therapeutic compound may be released to the at least one hypoxic cell after the composition is exposed to a release signal chosen from: ultrasound energy above an ultrasound release threshold, x-ray energy above an x-ray release threshold, contact with a release compound, a temperature above a temperature release threshold, a pH above or below a pH release threshold, and any combination thereof. Each of the at least one therapeutic compounds may be chosen from: a chemotherapy compound, a radiotherapy compound, and a sonotherapy compound; the radiotherapy compound may be hafnium oxide.

Another aspect of the present disclosure provides a method of detecting at least one hypoxic cell in a patient. The method includes forming at least one marked hypoxic cell by injecting an amount of a hypoxia-targeting agent into the patient, forming at least one contrast-enhanced hypoxic cell by injecting an amount of a composition comprising a plurality of functionalized microbubbles, and detecting at least one hypoxic cell by obtaining an image of the patient. The hypoxia-targeting agent may selectively bind to each hypoxic cell, and each marked hypoxic cell may include the hypoxia-targeting agent bound to each hypoxic cell. The plurality of functionalized microbubbles may include a selective binding compound attached to an exposed microbubble surface; the selective binding compound selectively binds to the hypoxia-targeting agent bound to each hypoxic cell; and each contrast-enhanced hypoxic cell includes one or more functionalized microbubbles attached to each marked hypoxic cell via a linkage between the hypoxia-targeting agent and the selective binding compound. At least one hypoxic cell may be detected as a high-contrast region within the image. The method may further include rupturing each functionalized microbubble using high-intensity ultrasound, obtaining a second image of the patient, and detecting the at least one hypoxic cell by comparing the image with the second image. The method may further include rupturing a portion of the functionalized microbubbles remaining in circulation after a threshold binding period using high-intensity ultrasound prior to obtaining the image of the patient. Each functionalized microbubble may have a microbubble diameter ranging from about 0.1 μm to about 10 μm. The selective binding compound may be chosen from an antibody with an antigenic determinant that is the hypoxia-targeting agent, a polypeptide, an organic molecule, and any combination thereof. The antibody may be conjugated with biotin, the microbubble surface may be coated with streptaviden, and the antibody may be attached to the microbubble surface via a biotin-streptaviden linkage in this method. The hypoxia-targeting agent may be chosen from an endogenous hypoxic cell marker and an exogenous hypoxia-targeting agent; the hypoxia-targeting agent may preferentially bind to the hypoxic cell. The hypoxia-targeting agent may be a compound, derivative, or metabolite of a compound chosen from: nitromidazole, misonidazole, and pimonidazole. The at least one hypoxic cell may be detected using an imaging method chosen from: ultrasound, MRI, x-ray scattering, and PET. The image of the patient may be further analyzed to identify one or more surgical planning data chosen from: a location of the hypoxic cells; a relative size of a tumor mass or other disorder associated with the hypoxic cells; a position of the hypoxic cells relative to surrounding structures such as tissues, organs, and bones; a severity of a disorder associated with the hypoxic cells; and any combination thereof.

This method overcomes the limitations of previous methods of detecting hypoxic tissues by selectively targeting hypoxic vascular and/or blood cells adjacent to the afflicted region associated with the hypoxic tissues. The vascular cells and blood cells are readily accessible by intravenous transfusions/injections, and the functionalized microbubbles may be detected using ultrasound or other non-invasive imaging technologies. As a result, this method provides a novel non-invasive method of detecting and/or treating hypoxic tissues associated with a variety of disorders including, but not limited to, tumor masses, cardiovascular disorders, and strokes.

Still another aspect of the present disclosure provides a method of treating at least one hypoxic cell in a patient. The method includes: forming at least one marked hypoxic cell by injecting an amount of a hypoxia-targeting agent into the patient, forming at least one contrast-enhanced hypoxic cell by injecting an amount of a composition comprising a plurality of functionalized microbubbles, detecting the at least one hypoxic cell by obtaining an image of the patient, and releasing the therapeutic compound to each contrast-enhanced hypoxic cell by exposing each microbubble to a release signal. The release signal may be chosen from: ultrasound energy above an ultrasound release threshold, x-ray energy above an x-ray release threshold, contact with a release compound, a temperature above a temperature release threshold, a pH above or below a pH release threshold, and any combination thereof. The hypoxia-targeting agent may selectively bind to each hypoxic cell and each marked hypoxic cell may include the hypoxia-targeting agent attached to each hypoxic cell. The functionalized microbubble may include a selective binding compound attached to an exposed microbubble surface and a therapeutic compound attached to the exposed microbubble surface or contained within each microbubble; the selective binding compound selectively binds to the hypoxia-targeting agent bound to each hypoxic cell; and each contrast-enhanced hypoxic cell comprises one or more functionalized microbubbles attached to each marked hypoxic cell via a linkage between the hypoxia-targeting agent and the selective binding compound. At least one hypoxic cell may be detected within as a high-contrast region within the image. Each microbubble has a microbubble diameter ranging from about 0.1 μm to about 10 μm. The selective binding compound may be chosen from an antibody with an antigenic determinant that is the hypoxia-targeting agent; a polypeptide; an organic molecule; and any combination thereof. The antibody may be conjugated with biotin, each microbubble surface may be coated with streptaviden, and the antibody may be attached to the microbubble surface via a biotin-streptaviden linkage. The hypoxia-targeting agent may be chosen from an endogenous hypoxic cell marker and an exogenous hypoxia-targeting agent; the hypoxia-targeting agent may preferentially bind to the hypoxic cell. The hypoxia-targeting agent may be a compound, derivative, or metabolite of a compound chosen from: nitromidazole, misonidazole, and pimonidazole. The at least one hypoxic cell is detected using an imaging method chosen from: ultrasound, MRI, x-ray scattering, and PET. The at least one therapeutic compound may be chosen from a chemotherapy compound, a radiotherapy compound, and a sonotherapy compound. The radiotherapy compound may include a high Z-element chosen from gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium. The method may further include analyzing the image of the patient to identify one or more surgical planning data chosen from: a location of the hypoxic cells; a relative size of a tumor mass or other disorder associated with the hypoxic cells; a position of the hypoxic cells relative to surrounding structures such as tissues, organs, and bones; a severity of a disorder associated with the hypoxic cells; and any combination thereof.

Other aspects and features of the disclosure are detailed below.

### DESCRIPTION OF THE DRAWINGS

The following drawings illustrate various aspects of the disclosure.

FIG. 1 is a graph schematically illustrating an experimental method of assessing the efficiency of targeting of hypoxic cells by functionalized microbubbles in an aspect.

FIG. 2 is a bar graph summarizing a comparison of the efficiency of targeting untreated hypoxic cells, PIMO-treated hypoxic cells, and PIMO-treated normoxic cells using functionalized microbubbles.

Corresponding reference characters and labels indicate corresponding elements among the views of the drawings. The headings used in the figures should not be interpreted to limit the scope of the claims.

### DETAILED DESCRIPTION

The present disclosure provides compositions and methods for detecting and/or treating hypoxic cells in the body. The composition may include a plurality of functionalized microbubbles with a bound selective binding compound and optionally a therapeutic compound. The composition may be used in a method of detecting and optionally treating hypoxic cells. A detailed description of the composition for detecting and treating hypoxic cells, as well as methods of detecting and treating hypoxic cells using various aspects of the composition, are provided herein below.

### (I) Composition for Detecting and Treating Hypoxic Cells

Hypoxia may be associated with certain tumors, vascular diseases, or stroke. Hypoxic cells may be highly resistant to chemotherapy, radiation therapy, and other methods of therapy. As a result, the detection of hypoxic areas may facilitate the planning of various treatments and/or implement delivery of targeted therapy.

In an aspect, the composition may include a plurality of functionalized microbubbles used in combination with ultrasound, MRI, or other compatible imaging techniques to detect and/or treat hypoxic cells. In an aspect, the functionalized microbubbles may detect a microenvironmental symptom of endothelial/blood tissues adjacent to tumor tissue or other disorders such as stroke or vascular disease. In another aspect, the functionalized microbubbles may include a selective binding compound attached to the microbubble surface via a linking compound. The functionalized microbubbles may further include a therapeutic compound for the treatment of a hypoxic region. The therapeutic compound may be attached to the surface with a linking compound or may be contained within the shell of the microbubble. The functionalized microbubbles may bind to exogenous markers/antigens bound to hypoxic cell membranes but the functionalized microbubbles may also bind to endogenous hypoxic cell markers displayed on the surface of hypoxic cell membranes.

The functionalized microbubbles may act as a contrast agent for variety of imaging methods. Non-limiting examples of suitable imaging methods include ultrasound, MRI, X-ray scattering, PET, SPECT, and any other known imaging modality, so long as the imaging method is compatible with the function of the contrast agent. In an aspect, the imaging method is ultrasound imaging. As an ultrasound contrast agent, the functionalized microbubbles may facilitate imaging by acoustic backscatter in an area in which the functionalized microbubbles have aggregated. In concert with hypoxic-targeting agents administered to mark hypoxic cells, the functionalized microbubbles may then be used to detect/target hypoxic endothelial/blood cells adjacent to afflicted tissues for use in the diagnosis and planning of treatment of a disorder associated with the hypoxic cells.

The composition may further act as a targeted delivery vehicle for therapeutic compounds including, but not limited to, radiotherapy drugs, radiosensitizing drugs, chemotherapy drugs, and other therapeutic compounds. Typically, hypoxic cells are associated with tumor tissue, but they may also be associated with vascular disease or stroke. The composition described herein may be used to detect and treat the hypoxic cells in and around these tissues.

### (a) Microbubbles

In an aspect, the composition includes a plurality of microbubbles. The microbubbles may function as an ultrasound contrast agent, but may also be used as a contrast agent for MRI and other imaging modalities. Microbubbles may typically include a gas core contained within an outer surface membrane constructed using materials including, but not limited to, proteins, lipids, or polymers. Non-limiting examples of gases suitable for inclusion within the microbubbles include air, carbon dioxide, nitrogen, oxygen, nitrous oxide, helium, argon, nitric oxide, xenon, a perfluorocarbon gas, and any mixture thereof. In other non-limiting aspects, the gas may include a fluorocarbon gas such as tetrafluoromethane, hexafluoroethane, octafluoropropane, decafluorobutane, perfluoro-isobutane and any combination thereof.

Ultrasound may be used to detect an aggregation of microbubbles through acoustic backscatter. Microbubbles are typically echogenic and may resonate at ultrasound frequencies in the range of about 1 MHz to about 10 MHz, eliciting a strong backscatter signal from the microbubbles. Depending on the ultrasound parameters, such as pulse magnitude, the microbubbles may also be ruptured through inertial cavitation. The rupture of microbubbles, typically accompanied by an induced shock wave, may be used to treat hypoxic cells by subjecting the hypoxic cells to the force of shock waves associated with microbubble rupture or the microbubbles may further release a therapeutic compound.

(i) Size Range

The diameter of the microbubbles may approximate the size of a red blood cell, resulting in comparable rheology characteristics in the microvessels and capillaries throughout the body. In an embodiment, the diameter of a microbubble may range from about 0.1 μm to about 20 μm. In other embodiments, the diameter of a microbubble may range from about 0.1 μm to about 1 μm, from about 0.1 μm to about 3 μm, from about 0.1 μm to about 5 μm, from about 1 μm to about 5 μm, from about 1 μm to about 15 μm, from about 1 μm to about 10 μm, from about 5 μm to about 10 μm, from about 5 μm to about 15 μm, and from about 10 μm to about 20 μm. In yet another embodiment, the diameter of a microbubble may be about 3 μm.

Without being limited to any particular theory, smaller microbubble diameters may facilitate the intravenous administration and convective transport of the microbubbles through the microvessels and capillaries in the body of the patient. In an aspect, a microbubble size of about less than 10 μm in diameter may be selected to approximate the characteristics of red blood cells. The microbubble diameter may also influence the intensity of the backscatter echo of the microbubbles.

(ii) Composition of Microbubbles

The shell, or outer surface membrane, of the microbubbles may be composed of surfactants, lipids, proteins, polymers, or any combination of these materials. Non-limiting examples of suitable proteins that may be used as a shell for the microbubbles include avidin, streptavidin, biotin, albumin, lysozyme, and any other suitable proteins known in the art. In an aspect, a streptavidin shell may allow for the linking of an antibody or other selective binding compound through a biotin linker. In another aspect, an avidin shell may allow for the linking of an antibody or other selective binding compound through a biotin linker. Non-limiting examples of suitable surfactants that may be used as a shell material for the microbubbles include SPAN-40, TWEEN-40, sucrose stearate, or any other surfactants known in the art. Non-limiting examples of suitable lipids that may be used as a shell material for the microbubbles include acyl lipids, glycoproteins, phospholipids, or any other suitable lipids known in the art. Non-limiting examples of suitable polymers that may be used as a shell material for the microbubbles include alginate, a double-ester polymer with ethylidene units, poly lactide-co-glycolide (PLGA), poly(vinyl alcohol) (PVA), polyperfluorooctyloxycaronyl-poly(lactic acid) (PLA-PFO), and any other suitable polymers known in the art. The microbubbles may be obtained commercially, or the microbubbles may be custom-made using any method known in the art.

The thickness of the outer layer of the microbubble may influence the functionality and responsiveness of the microbubble to ultrasound. Generally, the shell thickness of the microbubbles may range from about 5 nm to about 200 nm thick. In other aspects, the shell thickness of the microbubbles may range from about 5 nm to about 15 nm, from about 10 nm to about 25 nm, from about 20 nm to about 40 nm, from about 30 nm to about 50 nm, from about 40 nm to about 70 nm, from about 50 nm to about 90 nm, from about 70 nm to about 110 nm, from about 100 nm to about 130 nm, from about 120 nm to about 150 nm, from about 130 nm to about 170 nm, from about 150 nm to about 190 nm, and from about 170 nm to about 200 nm. In an aspect, the shell of the microbubbles may range from about 5 nm to about 15 nm in thickness.

Microbubbles with polymer shells typically have thicker shells than microbubbles with lipid or protein shells. Thicker shells may render the microbubble more resistant to compression and expansion, which may in turn influence echogenicity and drug delivery activity. In an aspect, a thinner shell material, such as a protein shell, may be used in the microbubbles and also deliver drugs to a targeted area. The zeta potential of the microbubble may further influence the stability of the microbubble or the delivery of the microbubble.

(iii) Concentration of Microbubbles in Composition

In an aspect, the functionalized microbubble composition is typically provided in the form of a suspension in a biocompatible solvent including, but not limited to, buffered saline solution. The suspension may include the functionalized microbubbles as well as additional solvents and/or diluents including, but not limited to fillers, emulsifiers, solubilizers, antioxidants, antimicrobials, and any other suitable diluent known in the art. In an aspect, the administration form may be an injectable suspension. In this aspect, the injectable suspension may include a biocompatible solvent including, but not limited to sterile buffered saline solution.

In one aspect, the injectable suspension of functionalized microbubbles may have a concentration of microbubbles ranging from about 10⁷ microbubbles/mL to about 10¹⁰ microbubbles/mL. In another aspect, the injectable suspension of functionalized microbubbles may have a concentration of microbubbles ranging from about 10⁸ microbubbles/mL to about 10¹⁰ microbubbles/mL.

In yet another aspect, the volume of functionalized microbubble composition administered to the patient may vary from about 0.1 mL to about 5 mL. If the functional microbubbles further include a therapeutic compound, the concentration of the functional microbubbles may be limited by the dosage of therapeutic compound that may be safely injected.

### (b) Selective Binding Compound

The composition may further include selective binding compounds attached to the surface of the microbubbles by a linking compound. The selective binding compound may be attached to the surface of a microbubble to target the microbubble to an exogenous or endogenous marker on the surface of a hypoxic cell or a cell near a hypoxic area. The selective binding compound may selectively bind to only hypoxic cells in an aspect. Hypoxic cells may be targeted by the binding compounds' affinity for binding either an endogenous marker unique to the hypoxic cell or to an exogenous hypoxia-targeting agent previously administered to the patient and bound to the hypoxic cells.

(i) Endogenous Hypoxia Markers

In an aspect, the selective binding compounds may selectively bind to endogenous hypoxia markers displayed on the surface of a hypoxic cell. In general, an endogenous hypoxia marker may be any molecule that is situated on the surface of a hypoxic cell at a significantly higher density than the corresponding density of the molecule on the surface of a normoxic cell. Typically, the synthesis and surface display of an endogenous hypoxia marker may be induced by the exposure of a cell to hypoxic conditions. In various aspects, the hypoxic conditions that induce the synthesis and surface display of an endogenous hypoxia marker may be acute, chronic, or any combination thereof. Any known hypoxia-induced surface protein, surface peptide or other hypoxia-induced surface compound may be selected as the endogenous hypoxia marker to which the selective binding compounds may preferentially bind. Non-limiting examples of suitable endogenous hypoxia markers include GLUT4 transporter protein, αv integrin, β1 integrin, ecto-5′-nucleotidase, urokinase-type plasminogen activator receptor (uPAR), carbonic anhydrase II (CAII), and carbonic anhydrase IX (CA IX).

(ii) Exogenous Hypoxia-Targeting Agents

In an aspect, the selective binding compounds may selectively bind to exogenous hypoxia-targeting agents. These exogenous hypoxia targeting agents may be administered to the patient prior to administering the composition. Examples of hypoxia-targeting agents may include, but are not limited to, nitromidazole, misonidazole, pimonidazole, and derivatives, analogs, and metabolites of these compounds. Additional non-limiting examples of hypoxia-targeting agents include 2(2-nitro-1H-imidazol-1-yl)-N-(2,2,3,3,3-pentafluoropropyl) acetamide; 2(2-nitro-1H-imidazol-1-yl)-N-(3,3,3-trifluoropropyl) acetamide; 3-nitro-10-methylbenzothiazolo[3,2-a]quinolinium chloride; 1-(5-Iodo-5-deoxy-beta-D-arabinofuranosyl)-2-nitroimidazole; heterocycle-N-oxide hypoxic cytotoxins; and derivatives, analogs and metabolites of these compounds.

The nitroheterocyclic compounds described above are known in the art as oxygen indicators. This class of compounds may provide a means of monitoring low oxygen partial pressures at a cellular level of resolution and may possess sufficient sensitivity to monitor hypoxic cells. Without being limited to any particular theory, the nitroheterocyclic compounds undergo bioreductive metabolism at a rate which increases substantially as the tissue's oxygen partial pressure decreases. This bioreductive metabolism results in the formation of reactive drug products which combine chemically to form adducts with predominantly cellular proteins. Because the metabolic binding of these compounds to cellular macromolecules is inhibited by oxygen, these compounds bind to hypoxic cells preferentially relative to normal healthy, oxygen-rich cells. This preferential metabolic binding, or adduct formation, provides a measure of the degree of hypoxia in a cell.

Pimonidazole (α-[(2-nitro-1H-imidazol-1-yl)methyl]-1-piperidineethanol) is a known radiosensitizer used to assess tumor hypoxia in carcinomas of the cervix, head, and neck. Pimonidazole is a nitromidazole compound that is reduced in hypoxic environments; reduced pimonidazole binds to thiol-containing proteins characteristic of hypoxic cells. The resulting complexes may accumulate in tissues and sensitize the cells within the tissue to be more susceptible to radiation treatment. This hypoxia marker has been widely used in experimental and clinical studies due to its chemical stability, water solubility, and wide tissue distribution characteristics. It is generally administered in an aqueous solution by injection or transfusion.

In an aspect, administration of the hypoxia-targeting agents may produce detectable cell surface antigens on hypoxic cells. The selective binding compounds may then be targeted to and bind with the cell surface antigens produced by the hypoxia-targeting agents bound to the hypoxic cells. In an aspect, the hypoxia-targeting agent may be a nitroimidazole. In another aspect, the hypoxia-targeting agent may be pimonidazole or any derivative thereof.

The hypoxia-targeting agent may be provided in any known form suitable for administration by the selected method. Non-limiting examples of suitable administration forms include tablets, capsules, sachets, injectable solutions, injectable suspensions, transdermal or transmucosal gels and lotions, and any other administration form known in the art. The administration form may include the hypoxia-targeting agent as well as additional solvents and/or diluents including, but not limited to fillers, emulsifiers, solubilizers, antioxidants, antimicrobials, and any other suitable diluent known in the art.

In an aspect, the administration form may be an injectable solution or suspension. In this aspect, the injectable solution or suspension may include a biocompatible solvent including, but not limited to sterile saline solution. In this aspect, if the hypoxia-targeting agent is pimonidazole, the injectable solution may include the pimonidazole hydrochloride dissolved in sterile saline solution at a concentration of up to about 116 mg/mL or about 400 millimolar, the solubility limit of pimonidazole hydrochloride in buffered saline solution.

The dosage of the hypoxia-targeting agent administered to the patient may vary depending on at least on factor including, but not limited to: the mode of administration; the species, mass, age and other characteristics of the patient; the chemical structure and properties of the hypoxia-targeting agent; the formulation of the dosage form of the hypoxia-targeting agent; and any other known factor related to dosing known in the art. In an aspect, if the hypoxia-targeting agent is pimonidazole in an injectable solution, as described herein above, the dosage may range from about 10 mg/kg to about 400 mg/kg. In other aspects, the dosage may range from about 20 mg/kg to about 40 mg/kg, from about 30 mg/kg to about 50 mg/kg, from about 40 mg/kg to about 60 mg/kg, from about 50 mg/kg to about 70 mg/kg, from about 60 mg/kg to about 80 mg/kg, from about 70 mg/kg to about 90 mg/kg, from about 80 mg/kg to about 100 mg/kg, from about 90 mg/kg to about 110 mg/kg, from about 100 mg/kg to about 175 mg/kg, from about 150 mg/kg to about 200 mg/kg, from about 175 mg/kg to about 225 mg/kg, from about 200 mg/kg to about 300 mg/kg, from about 250 mg/kg to about 350 mg/kg, and from about 300 mg/kg to about 400 mg/kg. In yet another aspect, the dosage may range from about 10 mg/kg to about 30 mg/kg. The dosage may be formulated to fall within a range bounded by a minimum dosage needed to ensure that sufficient hypoxia-targeting agent is available for binding to all hypoxic cells, but not exceeding a maximum dosage above which toxic effects and/or disruption of blood flow may occur.

For example, if the patient is a mouse and the hypoxia-targeting agent is pimonidazole, the dosage administered may be about 60 mg/kg. In another example, if the patient is a human and the hypoxia-targeting agent is pimonidazole, the dosage administered may be about 14 mg/kg, equivalent to about 0.5 g/m². In another example, if the patient is a dog and the hypoxia-targeting agent is pimonidazole, the dosage administered may be about 0.28 g/m².

The amount of injectable hypoxia-targeting agent solution may vary depending on at least one factor including, but not limited to: the desired dosage; the formulation of the injectable hypoxia-targeting agent solution; the solubility of the hypoxia-targeting agent in the solvent of the solution, and any other factor known in the art. For example, if the patient is a 70 kg human and the hypoxia-targeting agent is pimonidazole, the amount of pimonidazole administered may be about 8.5 mL [(14 mg/kg dosage×70 kg body mass)/(116 mg/mL solubility)].

(iii) Selective Binding Compounds

The selective binding compound may be any molecule capable of selectively binding exclusively to hypoxic cells. A microbubble may be targeted to a hypoxic cell which has been marked by a hypoxia-targeting agent. This targeting may be implemented by the affinity of the selective binding compound for binding the hypoxia-targeting agent. In one aspect, the selective binding compound may include, but is not limited to, an antibody with an antigenic determinant that is a hypoxia marker, a hypoxia-targeting agent, a cell surface antigen produced by the interaction of a hypoxia-targeting agent with the hypoxic cell and/or hypoxic environment surrounding the cell. In another aspect, the selective binding compound may include, but is not limited to a polypeptide, a non-peptide molecule, and any combination thereof. In an additional aspect, the selective binding compound may include an anti-pimonidazole antibody or an anti-pimonidazole binding molecule attached to the surface of the microbubble. In another additional aspect, the selective binding compound may include an anti-nitroimidazole antibody or an anti-nitroimidazole binding molecule attached to the surface of the microbubble.

The selective binding compound may be attached to the linking compound opposite to the microbubble surface. The linking compound may include, but is not limited to: a peptide, protein, a non-peptide molecule, or any other compound capable of connecting the shell of the microbubble and a selective binding compound. In an aspect, the linking compound may be biotin, which may attach to an avidin or streptavidin coated microbubble.

(iv) Concentration of Selective Binding Compounds in Composition

The selective binding compound may be included in the composition at a concentration on the microbubble surface that is capable of targeting the functionalized microbubbles to a hypoxic cell. In an aspect, sufficient pimonidazole concentrations may be produced on hypoxic cells to elicit both complement- and cell-mediated lysis of pimonidazole-labeled hypoxic cells. In an aspect, the concentration of the selective binding compound in the composition may be similar to the concentration of the hypoxia-targeting agent administered to the body.

### (c) Therapeutic Compound

The functional microbubbles may further include a therapeutic compound. The therapeutic compound may be a radiotherapy enhancing or hypoxia directed drug which may be loaded into or on the surface of the microbubble for implementing an additional treatment. Non-limiting examples of suitable therapeutic compounds include radiotherapy enhancing metal oxides, hafnium oxide, mitomycin C, porfiromycin, topotecan, efaproxiral, tirapazamine, nimorazole or other radiotherapy compounds, radiosensitizing compounds, chemotherapy compounds, or hypoxia directed drugs. The therapeutic compound may also be a radioactive substance for systemic radiation therapy in one non-limiting aspect. In another non-limiting aspect, the therapeutic compound may include radioactive iodine (¹³¹I) and other known radioactive substances for radiation therapy.

The concentration of the therapeutic compound may vary depending on the type of therapeutic drug and may be present in a concentration necessary to provide effective therapy to the hypoxic region but may not exceed levels that may be toxic to the patient.

### (II) Method for Detecting Hypoxic Cells

In an aspect, the composition described herein above may be used in conjunction with a method of detecting, and optionally treating, hypoxic cells in a patient. In this method, a hypoxia-targeting agent may be administered to the patient. This hypoxia-targeting agent may selectively bind to the hypoxic cells to form marked hypoxic cells. The cell-bound hypoxia-targeting agent may serve as a marker for the hypoxic cells to which the functionalized microbubbles may selectively bind.

A composition that includes a plurality of functionalized microbubbles, as described previously herein, may also be administered to the patient in this aspect. Each functionalized microbubble may include a microbubble with a selective binding compound attached to the microbubble surface via a linking compound. Upon administration, the selective binding compound may bind to the cell-bound hypoxia-targeting agent to form a contrast-enhanced marked hypoxic cell. As described previously above, each functionalized microbubble may function as a targeted contrast agent for subsequent imaging of the patient.

In this aspect, the hypoxic cells may be detected by obtaining an image of the patient using one or more of the imaging methods described herein above. Because the functionalized microbubbles, which are selectively attached to the hypoxic cells, may act as targeted contrast agents, the hypoxic cells may be detected as a high-contrast region within the obtained image. Depending on the type of image obtained, this image may be used to diagnose a disorder related to the hypoxic cells, assess the extent or severity of the disorder, and/or plan a treatment procedure to treat the disorder. Because the location and extent of the hypoxic cells may be determined from the obtained image, the procedure may be implemented in a targeted manner to reduce the exposure of the patient to noxious compounds such as radioactive compounds and/or chemotherapy compositions.

### (a) Hypoxic Cells Detected by Method

In an aspect, the hypoxic cell may be any living cell exposed to acute or chronic hypoxic conditions. For example, a hypoxic cell may be a cell within a tissue of a patient in which the blood supply to the tissue is compromised. In one aspect, the hypoxic cell may be an endothelial cell within a blood vessel associated with a tumor mass. Without being limited to any particular theory, it is thought that the notoriously high oxygen demands of tumor cells within the tumor mass exert chronically hypoxic conditions on these adjacent endothelial cells; as a result, tumor masses of various cancer types are stereotypically associated with these hypoxic endothelial cells. For example, between about 30% and about 67% of vascular endothelium cells associated with an SCK tumor mass may be detectibly hypoxic using various aspects of the method.

In this aspect, the detection of the hypoxic endothelial cells may provide diagnostic information regarding the location, size, level of activity, and oxidative state of the associated tumor mass. Because these endothelial cells are in direct contact with the circulating blood, the hypoxia-targeting agent and functionalized microbubbles may be introduced non-invasively via intravenous injection or transfusion.

Tumor masses associated with hypoxic endothelial cells may be result from any known cancer disorder. Non-limiting examples of cancer disorders that result in tumor masses associated with hypoxic endothelial cells include: brain cancer, breast cancer, ovarian cancer, testicular cancer, prostate cancer, bladder cancer, colon cancer, kidney cancer, liver cancer, lung cancer, and any other known cancer disorder. The percentage of endothelial cells associated with a tumor mass that are detectably hypoxic may vary due to a variety of factors including, but not limited to the size and activity of the tumor mass and the type of cancer.

### (b) Patient

In an aspect, the patient may be any mammalian organism. Non-limiting examples of mammalian organisms that are suitable patients in various aspects of the method include mammals from the Order Rodentia (mice); the Order Logomorpha (rabbits); the Order Carnivora, including Felines (cats) and Canines (dogs); the Order Artiodactyla, including Bovines (cows) and Suines (pigs); the Order Perissodactyla, including Equines (horses); and the Order Primates (monkeys, apes, and humans). In another aspect, the patient is a human.

### (c) Administration of Hypoxia-Targeting Agent

The hypoxia-targeting agent may be administered using any known method of administration of active compounds to mammalian patients. Non-limiting examples of suitable administration methods include oral administration, transdermal administration, transmucosal administration, intravenous injection or transfusion, intraarterial injection or transfusion, intraventricular injection, intraperitoneal injection, and intrathecal injection. In an aspect, the hypoxia-targeting agent is administered by intravenous injection or transfusion.

The hypoxia-targeting agent may be provided in any known form suitable for administration by the selected method. Non-limiting examples of suitable administration forms include tablets, capsules, sachets, injectable solutions, injectable suspensions, transdermal or transmucosal gels and lotions, and any other administration form known in the art. The administration form may include the hypoxia-targeting agent as well as additional solvents and/or diluents including, but not limited to fillers, emulsifiers, solubilizers, antioxidants, antimicrobials, and any other suitable diluent known in the art.

In an aspect, the administration form may be an injectable solution or suspension. In this aspect, the injectable solution or suspension may include a biocompatible solvent including, but not limited to sterile saline solution. In this aspect, if the hypoxia-targeting agent is pimonidazole, the injectable solution may include the pimonidazole hydrochloride dissolved in sterile saline solution at a concentration of up to about 116 mg/mL or about 400 millimolar, the solubility of pimonidazole hydrochloride in buffered saline solution.

The dosage of hypoxia-targeting agent administered to the patient may vary depending on at least one factor including, but not limited to: the mode of administration; the species, mass, age and other characteristics of the patient; the chemical structure and properties of the hypoxia-targeting agent; the formulation of the dosage form of the hypoxia-targeting agent; and any other known factor related to dosing known in the art. In an aspect, if the hypoxia-targeting agent is pimonidazole in an injectable solution, the dosage may range as described herein above. The dosage may be selected to fall with a range bounded by a minimum dosage needed to ensure that sufficient hypoxia-targeting agent is available for binding to all hypoxic cells, but not exceeding a maximum dosage above which toxic effects and/or disruption of blood flow may occur.

In an aspect, the injectable hypoxia-targeting agent solution may be injected into any suitable circulatory vessel including, but not limited to veins and arteries. Non-limiting examples of suitable circulatory vessels include a median cubital vein, any identifiable vein on the back of a hand of the patient, a femoral vein, a jugular vein, a carotid artery, a femoral artery, and any other suitable circulatory vessel for the injection of active compounds known in the art. In another aspect, the circulatory vessel may be selected to be immediately upstream of an area of interest in which hypoxic cells are thought to occur.

### (d) Method of Administering Functionalized Microbubble Composition

The functionalized microbubble composition may be administered using any known method of administration of active compounds in a solution or suspension form to mammalian patients. Non-limiting examples of suitable administration methods include intravenous injection or transfusion, intraventricular injection, intraperitoneal injection, and intrathecal injection. In an aspect, the functionalized microbubble composition is administered by intravenous injection or transfusion.

In an aspect, the functionalized microbubble composition may be provided in the form of a suspension in a biocompatible solvent such as buffered saline solution. The suspension form may include the functionalized microbubble described herein above as well as additional solvents and/or diluents including, but not limited to fillers, emulsifiers, solubilizers, antioxidants, antimicrobials, and any other suitable diluent known in the art. In an aspect, the administration form may be an injectable suspension. In this aspect, the injectable suspension may include a biocompatible solvent including, but not limited to sterile buffered saline solution.

The volume of functionalized microbubble composition administered to the patient may be selected based on at least one factor including, but not limited to: patient characteristics including species, size, age, and sex; the number of hypoxic cells to be detected; the concentration of functionalized microbubbles in the composition; the composition of the microbubbles; the size of the microbubbles; and the formulation of the functionalized microbubble composition including the amount of one or more therapeutic compounds included. For example, if a therapeutic compound is included in the functionalized microbubble composition, the maximum volume of composition administered to the patient may be limited to a volume corresponding to the maximum safe dosage of the therapeutic compound for the patient.

In an aspect, the functionalized microbubble composition is injected into any suitable circulatory vessel including, but not limited to veins and arteries. Non-limiting examples of suitable circulatory vessels include a median cubital vein, any identifiable vein on the back of a hand of the patient, a femoral vein, a jugular vein, a carotid artery, a femoral artery, and any other suitable circulatory vessel for the injection of active compounds known in the art. In another aspect, the circulatory vessel may be selected to be immediately upstream of an area of interest in which the hypoxic cells are thought to occur. In yet another aspect, the functionalized microbubble composition is injected into the same circulatory vessel used for the injection of the hypoxia-targeting agent solution.

### (e) Method of Obtaining Image of Patient

In an aspect, the hypoxic cells, which have been labeled with the functionalized microbubbles, may be detected by obtaining an image of the patient. Once an image is obtained, the hypoxic cells may be identified as a high contrast region within the image of the patient. Any suitable non-invasive imaging method may be used to obtain an image of the patient. Non-limiting suitable imaging methods include: ultrasound, MRI, X-ray scattering, PET, and any other suitable imaging method known in the art. In an aspect, ultrasound imaging may be used to obtain the image of the patient.

Without being limited to any particular theory, microbubbles are commonly used as a contrast agent for ultrasound imaging. Microbubbles are also used as a potentiating agent in ultrasound-related treatments such as sonothrombosis. Without being limited to any particular theory, the microbubbles are known to produce a back-scattered echo signal when insonified by ultrasound at relatively low acoustic pressures. The echogenicity, defined herein as the relative strength of the backscattered signal, may depend on the acoustic pressure and frequency of the ultrasound, as well as characteristics of the microbubbles. The echogenicity may reach a maximum when the ultrasound is applied at a microbubble resonance frequency. This microbubble resonance frequency may vary depending on the outside diameter and, to some extent, the elasticity or flexibility of the microbubble; larger and more elastic or flexible microbubbles may have a lower resonant frequency than smaller and less elastic or flexible microbubbles. Typically, the microbubble resonance frequency ranges from about 1 MHz to about 10 MHz for the majority of microbubble compositions and sizes commonly used.

In addition to acting as a contrast agent in ultrasound imaging, microbubbles also provide contrast, albeit through different physical mechanisms, for other imaging methods including, but not limited to, MRI, X-ray scattering, and PET imaging methods. To provide this contrast, the microbubbles may further incorporate additional contrast compounds including, but not limited to metallic nanoparticles, MRI contrast agents, radiomedical compounds, and any other suitable contrast compounds known in the art. These other contrast compounds may be attached to the microbubble surface, or may be contained within the microbubble in various aspects.

In an aspect, any known diagnostic ultrasound imaging device may be used to obtain the image of the patient. The particular type or model of the device may not be critical to the method of this disclosure. Devices designed for administering ultrasonic hyperthermia may also be used, including, but not limited to the devices described in U.S. Pat. Nos. 4,620,546, 4,658,828, and 4,586,512, the disclosures of each of which are hereby incorporated herein by reference in their entirety. The ultrasound imaging device may employ a resonant frequency (RF) spectral analyzer. The transducer probes may be applied externally or may be implanted. Ultrasound may be initiated at lower intensity and duration, and then intensity, pulse duration, and/or ultrasound frequency may be adjusted until the microbubbles are visualized on ultrasound. Complex waveforms, e.g. triangular, may be used to deliver mixed frequency ultrasound using a single transducer. Alternatively, two or more frequencies (mixed in specified proportions) may be used to drive a single transducer or sonic horn.

In an aspect, the patient image may be analyzed to detect the hypoxic cells. Because the hypoxic cells may have the functionalized microbubbles attached via a link between the selective binding compound and the hypoxia-targeting agent, the hypoxic cells may be detected as a region of high contrast within the patient image.

In another aspect, the functionalized microbubbles may be ruptured by exposing the microbubbles to a relatively high intensity of ultrasound at a frequency relatively near the microbubble resonance frequency, as described in detail herein below. Upon rupture, the functionalized microbubbles may no longer function as ultrasound contrast agents, as is known in the art. As a result, the region of high contrast previously associated with the labeled hypoxic cells may no longer be present in the second patient image. In this aspect, the second patient image may be compared to the original patient image obtained prior to rupturing the functionalized microbubbles; the hypoxic cells may be detected as the regions in which the ultrasound signals differ significantly between the original patient image and the second patient image.

In another aspect, the portion of functionalized microbubbles remaining in circulation after a threshold binding period may be ruptured by exposing the microbubbles to a relatively high intensity of ultrasound at a frequency relatively near the microbubble resonance frequency. Once the functionalized microbubbles remaining in circulation have been ruptured, an image of the patient may be obtained. In an aspect, the region of contrast within the patient image corresponds exclusively to the functionalized microbubbles bound to hypoxic cells. The threshold binding period, as used herein, refers to a length of time after the introduction of the composition into the circulatory vessel of the patient during which a concentration of functionalized microbubbles sufficient to detect and/or treat the hypoxic cells have bound to the hypoxic cells. After the threshold binding period has elapsed, any functionalized microbubbles remaining in circulation may be ruptured without compromising the efficacy of the detection and/or treatment of the hypoxic cells. In an aspect, the functionalized microbubbles remaining in circulation may be ruptured by applying the ultrasound to a region that is spatially separated from the region within which the hypoxic cells are thought to reside. For example, the ultrasound may be applied to one or more appendages of the patient, including, but not limited to an arm and a leg of the patient.

### (f) Method of Treating Hypoxic Cells—Treatment Planning

The patient image obtained to detect the hypoxic cells may be further analyzed to inform and/or implement one or more treatments of the hypoxic cells and/or the disorder associated with the hypoxic cells. In an aspect, the patient image may be analyzed to provide information to be used in planning a treatment of the patient. Non-limiting examples of information provided by the analysis of the patient image include: the location of the hypoxic cells; relative size of the tumor mass or other disorder associated with the hypoxic cells; the position of the hypoxic cells relative to surrounding structures such as tissues, organs, and bones; and the severity of the disorder associated with the hypoxic cells.

For example, the severity of the disorder may be determined from a number of characteristics including, but not limited to: the number and relative separation of multiple tumor masses or other disorders associated with the hypoxic cells; the density of the hypoxic cells within a region; the regularity of the margin of a tumor mass associated with the hypoxic cells; and any other available diagnostic feature known in the art.

The information provided by the patient image may further be used to determine an appropriate type of treatment and/or dosage to treat the hypoxic cells and associated tumor mass or other disorder. The precision of the information provided by the patient image may allow a more precise dosage to be used in the treatment, because the treatment will be targeted precisely to the region of the disorder. As a result, a comparable response to a treatment may be obtained using a lower than typical dosage, or an enhanced response may be obtained using a typical response, particularly if the treatment dosage is limited by toxicity or other non-therapeutic considerations.

### (g) Method of Treating Hypoxic Cells—Targeted Treatment

The functionalized microbubbles may include one or more therapeutic compounds in various aspects, as described previously herein. Each therapeutic compound may be attached to the microbubble surface or may be contained within the microbubble. In an aspect, the therapeutic compound may be attached to the microbubble using a linkage that is sensitive to a release signal. Alternatively, each therapeutic compound contained within the microbubble may be released in response to a release signal. Non-limiting examples of suitable release signals may include: ultrasound energy above an ultrasound release threshold; x-ray energy above an x-ray release threshold; contact with a release compound; contact with a cleaving enzyme; a temperature above a temperature release threshold; and a pH above or below a pH release threshold.

In one aspect, the therapeutic compound may be released by the functionalized microbubbles in response to ultrasound above an ultrasound release threshold. In this aspect, the ultrasound pulses may be delivered at a frequency near the microbubble resonance frequency and an ultrasound intensity sufficient to induce a stable oscillation. The shear forces and local convective mixing resulting from the oscillating microbubble may facilitate the delivery of therapeutic compounds. In this aspect, the ultrasound release threshold may be the minimum ultrasound intensity that results in the stable oscillation of the functionalized microbubble.

In another aspect, the therapeutic compound may be released by the functionalized microbubbles upon the rupture of the microbubbles in response to ultrasound delivered at a relatively high intensity. In this aspect, the ultrasound pulses may be delivered at a frequency near the microbubble resonance frequency and an ultrasound intensity sufficient to induce the rupture of the microbubble. The shear forces and local convective mixing resulting from the rupture of the microbubble may facilitate the delivery of therapeutic compounds. If the therapeutic compounds are contained within the microbubble, the rupture of the microbubble may result in the release of the therapeutic compound.

In yet another aspect, the therapeutic compound may be released in response to a release signal other than ultrasound. For example, the functionalized microbubbles may include a radiotherapeutic compound including, but not limited to, a high-Z element. In this example, the high-Z element may release a plurality of electrons in response to interaction with X-rays; the release signal in this example may be the X-ray radiation above an X-ray threshold value representing the minimum X-ray intensity needed to generate electrons from the high-Z element. Non-limiting examples of suitable high-Z elements for radiation enhancement include: gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium.

In another example, the therapeutic compound may be attached to the microbubble via a linker compound. In this example, the linker compound may be vulnerable to cleavage due to exposure to certain physical or chemical conditions. These physical or chemical conditions may function as the release signal including, but not limited to: contact with a cleaving enzyme; a temperature above a temperature release threshold; and a pH above or below a pH release threshold.

The linker compound may be vulnerable to cleavage by a particular cleaving enzyme. This cleaving enzyme may be introduced into the region of the hypoxic cells by any known means including, but not limited to, injection or transfusion in a manner similar to the injection or transfusion of the functionalized microbubble composition described previously herein. Alternatively, the cleaving enzyme may be situated within the microbubble and may be released upon exposure to ultrasound radiation above the ultrasound threshold, causing the cleavage of a linker group binding a therapeutic compound to the surface of the microbubble.

The linker may be vulnerable to spontaneous degradation or cleavage upon exposure to other physical and chemical conditions including, but not limited to, elevated temperature and increases or decreases in pH. For example, the therapeutic compounds may be released upon exposure of the functionalized microbubbles to elevated temperatures. The elevated temperatures may be generated by the application of a heating pad or other heating device, or as a result of exposure to infrared radiation. Alternatively, the elevated temperature may be locally elevated by the exposure of the functionalized microbubbles to ultrasound at a frequency and intensity sufficient to induce local heating.

Any therapeutic compound may be used in conjunction with the treatment of the hypoxic cells and associated disorders. Non-limiting examples of suitable therapeutic compounds are described previously herein. In one aspect, the therapeutic compound may address the condition of hypoxic vascular endothelial cells associated with a tumor mass. As discussed previously herein, these hypoxic endothelial cells are relatively resistant to the effects of radiation, thereby diminishing the desired effect of killing the cells of the tumor mass. In this aspect, the therapeutic compound may enhance the oxygenation status of the hypoxic vascular endothelial cells, thereby enhancing the effects of radiation therapy on these cells and by extension the cells of the tumor mass. Alternatively, the therapeutic compound may ablate, kill, or otherwise incapacitate the vascular endothelial cells, thereby eliminating their ability to absorb therapeutic radiation.

In another aspect, the therapeutic compound may be a radiotherapeutic compound that generates and releases radioactive particles in the immediate vicinity of the hypoxic vascular cells and the associated cells of the tumor mass. In this aspect, the radiotherapeutic source is situated in intimate contact with the target cells and tissues. As a result, the desired therapeutic effect may be achieved with a relatively low amount of the radiotherapeutic compound compared to existing radiotherapeutic treatment methods.

In yet another aspect, the functionalized microbubbles may include a single therapeutic compound. In an additional aspect, the functionalized microbubbles may include two or more therapeutic compounds having similar therapeutic effects; for example, the functionalized microbubbles may include two different chemotherapy compounds. In another additional aspect, the functionalized microbubbles may include two or more therapeutic compounds having different therapeutic effects. For example, the functionalized microbubbles may include a therapeutic compound that enhances the oxidative state of the hypoxic cells as well as a second radiotherapeutic compound that produces radioactive products to kill the surrounding cells. In this example, the functionalized microbubbles may include two therapeutic compounds: a first compound to lower the resistance of the surrounding cells to the effects of the second compound.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### EXAMPLES

The following examples are included to demonstrate preferred embodiments of the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the specific embodiments that are disclosed and still obtain a like or similar result without departing from the spirit and scope of the disclosure, therefore all matter set forth is to be interpreted as illustrative and not in a limiting sense.

### Example 1

### In Vivo Targeting of Mouse Breast Tumor Using Anti-Pimonidazole Antibody-Bound Microbubbles

To assess the targeting efficacy of the functionalized microbubbles, the following experiments were conducted. Functionalized microbubbles were produced that included an anti-pimonidazole antibody as the selective binding compound bound to the surface of streptavidin-coated microbubbles. The functionalized microbubbles were introduced into a circulatory vessel supplying a 4T1 mouse breast tumor that had been pre-treated by prior introduction of pimonidazole into the circulatory vessel, or had received no pre-treatment (control). In addition, microbubbles were similarly introduced into a circulatory vessel supplying a mouse muscle.

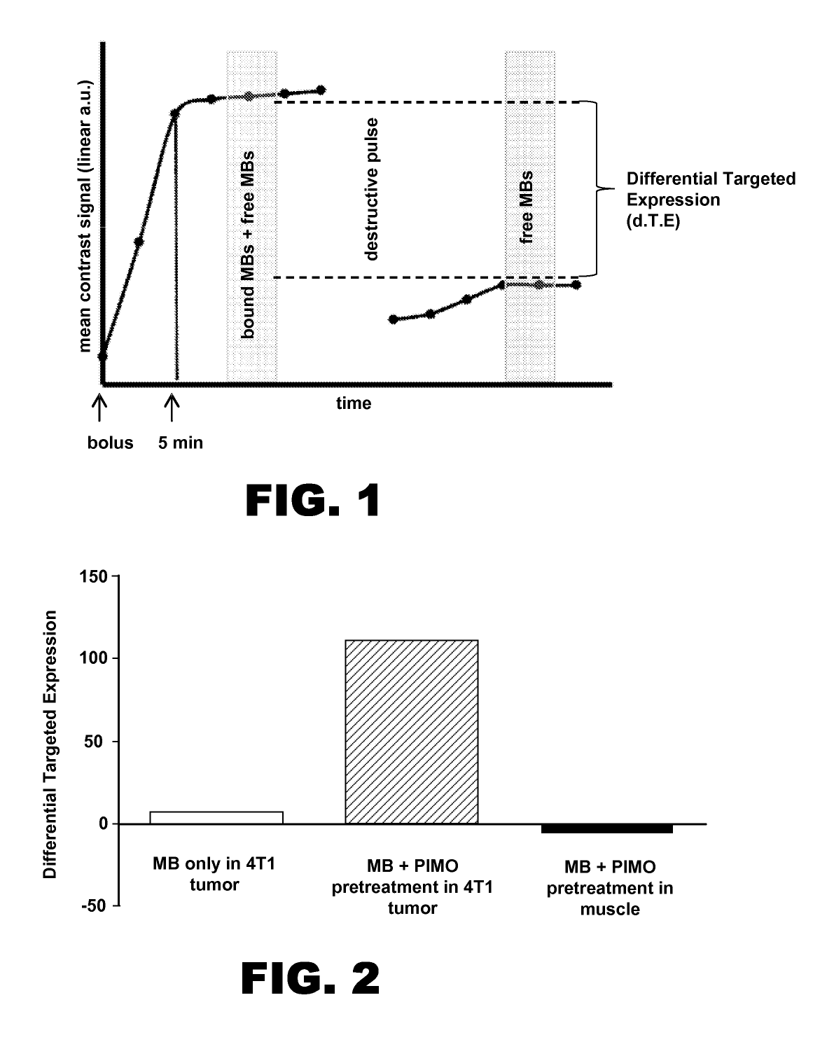

To assess the contrast enhancement induced by the treatment with the functionalized microbubbles, the following procedure, summarized schematically in FIG. 1, was performed. A bolus of functionalized microbubbles in suspension was introduced into a circulatory vessel supplying a 4T1 mouse breast tumor or mouse muscle. About 6 minutes after the functionalized microbubbles were introduced, a region in the vicinity of the 4T1 tumor or mouse muscle was subjected to ultrasound imaging and the mean contrast signal within each region was calculated. The region was then subjected to a high-intensity ultrasound pulse to burst the functionalized microbubbles, and the identical regions were then subjected to a second round of ultrasound imaging and calculation of mean contrast signal. The difference between the mean contrast signal before and after exposure to the high intensity pulse within each imaging region was calculated to determine the differential Targeted Expression (d.T.E.) used to quantify the efficiency of targeting the 4T1 tumor cells and adjacent cells such as vascular endothelial cells associated with the circulatory vessel supplying the 4T1 tumor.

The results of the contrast mean power calculations described above are summarized in FIG. 2. The introduction of the functionalized microbubbles into the blood vessel supplying a 4T1 mouse breast tumor that had been pre-treated with pimonidazole resulting in a 17-fold increase in the d.T.E. of the microbubbles within the 4T1 tumor compared to the untreated 4T1 tumor. When the pimonidazole hypoxia-targeting agent was absent from the 4T1 tumor tissue, the targeted microbubbles bound in relatively minute amounts. Similarly, the targeted microbubbles failed to bind to the muscle tissue.

In addition, the data obtained above were analyzed to estimate the percentage of cells targeted by the functionalized microbubbles. The percentage difference between the contrast signal before and after the high intensity pulse was calculated to estimate the percentage of the cells in the region targeted by the functionalized microbubbles. For the 4T1 tumors analyzed using the methods described herein above, the percentage of cells in contact with the circulation in the 4T1 tumors that were targeted by the functionalized microbubbles ranged between about 29% and about 67% of all cells within the region. In a separate measurement using flow cytometry (not shown), about 32% of the cells in contact with the circulation in the 4T1 tumors were targeted by the functionalized microbubbles.

The results of this experiment demonstrated the feasibility of using functionalized microbubbles in conjunction with a hypoxia-targeting agent to detect hypoxic cells in a tumor mass.

1. A targeted contrast composition for the detection of at least one hypoxic cell, the composition comprising a plurality of functionalized microbubbles, wherein each functionalized microbubble comprises:
(a) a microbubble comprising an exposed microbubble surface;
(b) a linking compound attached to the microbubble surface, the linking compound chosen from: streptaviden, avidin, neutravidin, and any combination thereof; and
(c) a selective binding compound attached to the linking compound opposite to the microbubble surface, the selective binding compound chosen from an antibody with an antigenic determinant that is a hypoxia-targeting agent; a polypeptide; a non-peptide molecule; and any combination thereof;
wherein the binding compound selectively binds to the hypoxia-targeting agent attached to the hypoxic cell and the hypoxia-targeting agent is chosen from an endogenous hypoxic cell marker and an exogenous hypoxia-targeting agent comprising a compound, derivative, or metabolite of a compound chosen from: nitromidazole, misonidazole, pimonidazole, and any combination thereof.

2. The composition of claim 1, wherein the microbubble has a microbubble diameter ranging from about 0.1 μm to about 10 μm.

3. The composition of claim 1, wherein the linking compound is streptaviden and the selective binding compound is the antibody, wherein:
(a) the antibody is conjugated with biotin; and
(b) the antibody is attached to the microbubble surface via a biotin-streptaviden linkage.

4. The composition of claim 1, wherein the at least one hypoxic cell is detected using an imaging method chosen from: ultrasound, MRI, x-ray scattering, and PET.

5. The composition of claim 1, wherein each functionalized microbubble further comprises at least one therapeutic compound, wherein:
(a) each therapeutic compound is attached to the microbubble surface, attached to the linking compound, attached to the selective binding compound, or contained within the microbubble; and
(b) each therapeutic compound is released to the at least one hypoxic cell after the composition is exposed to a release signal chosen from: ultrasound energy above an ultrasound release threshold, x-ray energy above an x-ray release threshold, contact with a release compound, a temperature above a temperature release threshold, a pH above or below a pH release threshold, and any combination thereof.

6. The composition of claim 5, wherein each of the at least one therapeutic compounds is chosen from: a chemotherapy compound, a radiotherapy compound, and a sonotherapy compound.

7. The composition of claim 6, wherein the radiotherapy compound is hafnium oxide.

8. A method of detecting at least one hypoxic cell in a patient, the method comprising:
(a) forming at least one contrast-enhanced hypoxic cell by injecting an amount of a composition comprising a plurality of functionalized microbubbles into the patient, wherein:
i. the functionalized microbubble comprises a selective binding compound attached to an exposed microbubble surface, the selective binding compound chosen from an antibody with an antigenic determinant that is a hypoxia-targeting agent, a polypeptide, an organic molecule, and any combination thereof;
ii. the selective binding compound selectively binds to the hypoxia-targeting agent bound to each hypoxic cell, the hypoxia-targeting agent chosen from an endogenous hypoxia-targeting agent and an exogenous hypoxia-targeting agent; and
iii. each contrast-enhanced hypoxic cell comprises one or more functionalized microbubbles attached to each marked hypoxic cell via a linkage between the hypoxia-targeting agent and the selective binding compound; and
(b) detecting the at least one contrast-enhanced hypoxic cell by obtaining an image of the patient, wherein the at least one contrast-enhanced hypoxic cell is detected as a high-contrast region within the image.

9. The method of claim 8, wherein the antibody is conjugated with biotin, the microbubble surface is coated with streptaviden, and the antibody is attached to the microbubble surface via a biotin-streptaviden linkage.

10. The method of claim 8, further comprising rupturing each functionalized microbubble using high-intensity ultrasound, obtaining a second image of the patient, and detecting the at least one hypoxic cell by comparing the image with the second image.

11. The method of claim 8, further comprising rupturing a portion of the functionalized microbubbles remaining in circulation after a threshold binding period using high-intensity ultrasound prior to obtaining the image of the patient.

12. The method of claim 8, further comprising forming at least one marked hypoxic cell prior to forming the at least one contrast-enhanced hypoxic cell by injecting an amount of the exogenous hypoxia-targeting agent into the patient, wherein:
(a) the exogenous hypoxia-targeting agent is chosen from a compound, derivative, or metabolite of a compound chosen from: nitromidazole, misonidazole, and pimonidazole;
(b) the hypoxia-targeting agent selectively binds to each hypoxic cell; and
(c) each marked hypoxic cell comprises the hypoxia-targeting agent bound to each hypoxic cell.

13. The method of claim 8, wherein the at least one hypoxic cell is detected using an imaging method chosen from: ultrasound, MRI, x-ray scattering, and PET.

14. The method of claim 8, wherein the image of the patient is further analyzed to identify one or more surgical planning data chosen from: a location of the hypoxic cells; a relative size of a tumor mass or other disorder associated with the hypoxic cells; a position of the hypoxic cells relative to surrounding structures such as tissues, organs, and bones; a severity of a disorder associated with the hypoxic cells; and any combination thereof.

15. A method of treating at least one hypoxic cell in a patient, the method comprising:
(a) forming at least one contrast-enhanced hypoxic cell by injecting an amount of a composition comprising a plurality of functionalized microbubbles, wherein:
i. the functionalized microbubble comprises a selective binding compound attached to an exposed microbubble surface and a therapeutic compound attached to the exposed microbubble surface or contained within each microbubble;
ii. the selective binding compound is chosen from an antibody with an antigenic determinant that is a hypoxia-targeting agent, a polypeptide, an organic molecule, and any combination thereof;
iii. the hypoxia-targeting agent is chosen from an endogenous hypoxia-targeting agent and an exogenous hypoxia-targeting agent;
iv. the selective binding compound selectively binds to the hypoxia-targeting agent bound to each hypoxic cell; and
v. each contrast-enhanced hypoxic cell comprises one or more functionalized microbubbles attached to each marked hypoxic cell via a linkage between the hypoxia-targeting agent and the selective binding compound;
(b) detecting the at least one contrast-enhanced hypoxic cell by obtaining an image of the patient, wherein the at least one contrast-enhanced hypoxic cell is detected as a high-contrast region within the image; and
(c) releasing the therapeutic compound to each contrast-enhanced hypoxic cell by exposing each microbubble to a release signal.

16. The method of claim 15, wherein the at least one hypoxic cell is detected using an imaging method chosen from: ultrasound, MRI, x-ray scattering, and PET.

17. The method of claim 15, wherein the release signal is chosen from: ultrasound energy above an ultrasound release threshold, x-ray energy above an x-ray release threshold, contact with a release compound, a temperature above a temperature release threshold, a pH above or below a pH release threshold, and any combination thereof.

18. The method of claim 15, wherein each of the at least one therapeutic compounds is chosen from a chemotherapy compound, a radiotherapy compound, and a sonotherapy compound.

19. The method of claim 18, wherein the radiotherapy compound comprises a high Z-element chosen from gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium.

20. The method of claim 15, further comprising forming at least one marked hypoxic cell prior to forming the at least one contrast-enhanced hypoxic cell by injecting an amount of the exogenous hypoxia-targeting agent into the patient, wherein:
(a) the exogenous hypoxia-targeting agent is chosen from a compound, derivative, or metabolite of a compound chosen from: nitromidazole, misonidazole, and pimonidazole;
(b) the hypoxia-targeting agent selectively binds to each hypoxic cell; and
(c) each marked hypoxic cell comprises the hypoxia-targeting agent bound to each hypoxic cell.
